(19) United States
(12) Patent Application Publication
(10) Pub. No.: US 2005/0226814 A1
(43) Pub. Date: Oct. 13, 2005

(54) Diagnostic method and kit for implantation of a sustained release drug-delivery implant with a steroid

(75) Inventor: Brian Levy, Rochester, NY (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/093,122

(22) Filed: Mar. 29, 2005

Related U.S. Application Data
(60) Provisional application No. 60/561,866, filed on Apr. 13, 2004.

(51) Int. Cl. A61K 49/00

(52) U.S. Cl. 424/9.1

(57) ABSTRACT

The present invention is a method of the present invention screens a patient with macular edema associated with visual loss for implantation of a sustained release drug-delivery implant that is less invasive than implanting a drug-delivery implant and is minimally invasive. The method comprises injecting a first steroid, eg. triamcinolone acetonide, having anti-inflammatory activity as a bolus into a patient's eye. Patients that experience a reduction in swelling in the retina (typically the macula and preferably the fovia) and improved vision are identified as candidate patients. Candidate patients are patients that are more likely to benefit from the implantation of a sustained release drug-delivery implant into the eye containing a second steroid, for example fluocinolone acetonide.

### BACKGROUND OF THE INVENTION

1. Field of the Invention

This invention relates to the diagnosis of patients with macular edema and more particularly to the identification of patients that are likely to have visual improvement with the implantation of a sustained release drug-delivery implant with a steroid.

2. Discussion of the Related Art

The macula is the portion of the retina that is responsible for approximately the central 15 degrees of vision. Macular edema is the thickening of the macula and is typically caused when fragile capillaries and microaneurysms in the retina leak fluid. Early in the development of this disease, patients with macular edema may still have normal vision. However, as the condition worsens, it may cause blurred and distorted vision. Eventually, permanent vision loss will occur.

Standard treatment for macular edema includes focal laser photocoagulation. According to this procedure, a fluorescein angiogram identifies the areas in the retina where the vessels in the retina leak fluid. Laser treatment is used to seal leaking vessels and reduce swelling of the macula. Care must be taken during laser surgery to avoid the highly sensitive fovia or the surgery could possibly damage central vision.

The risk of visual loss for patients with macular edema who undergo focal laser photocoagulation is reduced by more than 50%; however, laser treatment does not usually improve vision. Furthermore, multiple laser surgery is frequently required to resolve the swelling.

Treatment of disorders in the retinal area by systemic drug therapy is difficult because very little drug passes through the blood-retinal barrier. Thus, treatment of the retinochoroidal disorders by an intravenous injection or oral administration is ineffective.

It is already known that steroids are useful medicament for several retinochoroid disorders when administered directly into the eye of a patient. See U.S. Pat. No. 5,770,589 (treatment of macular degeneration by triamcinolone injection into a vitreous body), American Journal of Ophthalmology, vol. 89, pp. 131-136 (1980) (injected triamcinolone acetonide is believed to be useful for treatment of proliferative vitreoretinopathy) and EP Publ. No. 1380302 (betamethasone and hydrocortisone injected into the sub-conjuctival area inhibited proliferative vitreoretinopathy and was believed to effectively treat macular edema). However, injection of trimacinolone and trimacinolone acetonide into the eye of a patient did not have a significantly long-term effect.

Recently, Bausch & Lomb conducted clinical trials to assess the safety and efficacy of a long-term drug-delivery implant containing fluocinolone acetonide, a steroid with anti-inflammatory properties, for treatment of diabetic macular edema (DME). The implant had a tiny drug reservoir designed to deliver sustained and consistent levels of the steroid directly to the back of the eye for up to three years.

After thirty-four weeks, there was a statistically significant improvement in visual acuity in the implanted eyes. Comparative group with no implant had no statistically significant improvement in mean visual acuity in the fellow eyes. A large number of patients demonstrated an improvement in vision of three or more lines. However, not all patients experienced an improvement in vision. While implantation is desirable if a patient experiences an improvement in vision, surgical treatment may be preferred by the doctor or patient if there is no improvement in vision resulting from the administration of the steroid to avoid the invasive nature of the implant, risk of infection and side effects.

While significant improvements have been made in the treatment of macular edema, there exists a need to develop a way of predicting which patients will benefit from a sustained release drug-delivery implant by experiencing an improvement in vision that is less invasive than implanting the drug. The present invention addresses this and other needs.

### SUMMARY OF THE INVENTION

The method of the present invention screens a patient with macular edema associated with visual loss for implantation of a sustained release drug-delivery implant that is less invasive than implanting a drug-delivery implant and is minimally invasive. The method comprises injecting a first steroid, eg. triamcinolone acetonide, having anti-inflammatory activity as a bolus into a patient's eye. Patients that experience a reduction in swelling in the retina (typically the macula and preferably the fovia) and improved vision are identified as candidate patients. Candidate patients are patients that are more likely to benefit from the implantation of a sustained release drug-delivery implant into the eye containing a second steroid, for example fluocinolone acetonide. Generally, the swelling in the macula of the eye is measured by ocular coherence tomography.

In one embodiment, there is a method of treating a candidate patient with macular edema comprising implanting a candidate patient with a sustained release drug delivery implant containing a second steroid, typically fluocinolone acetonide. The candidate patient is evaluated by injecting a first steroid having anti-inflammatory activity (eg. triamcinolone actetonide) as a bolus into a patient's eye. Patients that experience (1) improved vision and (2) a reduction in swelling in the retina (typically the macula and preferably the fovia) are identified as a candidate patient.

In another embodiment, there is a kit for screening a patient with macular edema for use of a sustained-release drug-delivery implant containing a first steroid. The kit comprises a fluid injection device having a fluid reservoir containing a first steroid, for example triamcinolone acetonide, and a cannula that is configured to be inserted into the vitreous cavity of a patient's eye. The fluid injection device also has a compression device that is configured to move the first steroid from the reservoir through the cannula. Additionally, the kit comprises instructions to inject a bolus of the first steroid into a patient's eye and identify a patient that has a reduction in swelling of tissue in the eye and improved vision as a candidate patient for implantation of a sustained release, drug-delivery implant that contains a steroid with anti-inflammatory properties, such as flucinolone acetonide. Other advantages and features will be apparent from the below detailed description of the invention and examples.

### DETAILED DESCRIPTION OF THE INVENTION

Introduction

The present invention is a minimally invasive method that screens a patient with macular edema associated with visual loss to determine whether the patient will respond to treatment by implantation of a sustained release drug-delivery implant. The method comprises injecting a first steroid, eg. triamcinolone acetonide, having anti-inflammatory activity as a bolus into a patient's eye. Patients that experience a reduction in swelling in the retina (typically the macula and preferably the fovia) and improved vision are identified as candidate patients. Candidate patients are patients that are more likely to benefit from the implantation of a sustained release drug-delivery implant into the eye containing a second steroid, for example fluocinolone acetonide. The present invention, in one embodiment also includes a method of treating a patient identified by one or more of the diagnostic or screening methods disclosed above.

Definitions

“Drug” and “pharmaceutical” as used in this application are synonymous, are used interchangeably and are defined as any pharmaceutically acceptable compound that obtains a diagnostic effect or a local or systemic physiological or pharmacological effect. Reference to any particular drug includes without limitation its pharmaceutically acceptable salt, prodrug such as an ester or an ether, a salt of a prodrug, a solvate such as ethanolate or other derivative of such pharmacologically active drug. Generally, the synthesis and use of salts, prodrugs, salts of prodrugs, solvates and other derivatives are known in the art.

“Mixtures thereof” as used in this application to refer to mixtures of two or more drugs include mixtures of two or more drugs, esters, prodrugs, salts, solvates and/or other derivatives in any combination that is pharmaceutically acceptable.

“Salt” as it refers to the salt of a drug in this application is defined according to its technical meaning in the chemical art and not its ordinary dictionary meaning. Salts of drugs are derived, in one embodiment, from either inorganic or organic acids or bases. Examples of inorganic acids that are combined with a drug to make a salt include hydrochloric acid, hydrobromic acid, sulfuric acid, nitric acid, perchloric acid, and phosphoric acid. Examples of bases that are combined with a drug to make a salt include alkali metal (e.g., sodium) hydroxides, alkaline earth metal (e.g., magnesium) hydroxides, ammonia, and bases of formula NW₄⁺, wherein W is either a hydrogen substituent or a C₁₋₄ alkyl.

Examples of organic salts include: acetate, propionate, butyrate, hexanoate, heptanoate, undecanoate, palmoate, cyclopentanepropionate, adipate, alginate, aspartate, benzoate, citrate, oxalate, succinate, tartarate, lactate, maleate, filmarate, camphorate, nicotinate, pectinate, picrate, pivalate, tosylate, gluconate, digluconate, hemisulfate, methanesulfonate, ethanesulfonate, 2-hydroxyethanesulfonate, dodecylsulfate, camphorsulfonate, benzenesulfonate, 2-naphthalenesulfonate, thiocyanate, phosphate, glycerophosphate, and phenylpropionate.

“Derivative” as it refers to a drug in this application is defined as a chemically modified compound wherein the chemical modification takes place at one or more substituent groups and/or ring structures (including aromatic, alicyclic, or heterocyclic ring structures) of the drug while retaining at least a portion of the pharmacological activity of the drug from which it is derived.

“Prodrug” as it is used in this application to refer to the prodrug of a pharmaceutical is defined as a chemical precursor of a drug wherein the precursor itself either is or is not pharmacologically active but, upon administration to an animal, will be converted, either metabolically or otherwise, into a drug. Several prodrugs have been prepared and disclosed for a variety of drugs. See, for example, Bundgaard, H. and Moss, J., J. Pharm. Sci. 78: 122-126 (1989).

“Polymorphs,” “Isomers” and “Anomers” as they are used in this application to refer to polymorphs, isomers and anomers of a drug are defined according to their generally accepted meaning in the chemical art. Polymorphs, isomers (including stereoisomers, geometric isomer and optical isomers) and anomers of drugs described herein fall within the definition of the term drug.

“Pharmaceutically acceptable” as used herein to refer to any composition, compound, mixture, formulation, substance, etc is defined as any composition, compound, mixture, formulation, substance, etc that has diagnostic effect or a local or systemic physiological or pharmacological effect and where the side effects, level of purity and other qualities do not prohibit use of the same in an animal, preferably a patient, more preferably a human, most preferably a human patient.

“Steroid” as used in this application is defined as any hydrocarbon compound containing a seventeen-carbon, four-ring, cyclopentanonphenathrene nucleus system. For more guidance on types of steroids, see CRC Handbook of Chemistry and Physics, 75^{th} Edition, C.R.C Press, Boca Raton, Ch. 7, pp. 5-21 (1995). Pharmaceutically acceptable steroids are drugs and pharmaceuticals.

“First Steroid” and “Second Steroid” are defined as steroids. The designations “first” and “second” are arbitrary designations to refer to the use of a steroid in a particular step as defined by the claims and nothing else. “First steroid” and “Second steroid” can be the same steroid medicament or different steroid medicaments depending solely on the scope of the claims.

“Patient” as used in this application is defined as an animal patient that suffers from macular edema related vision loss. Preferably, the animal patient is a human patient.

“Macular edema related vision loss” as used in this application is defined as vision loss due to inflammation of the macula.

“Anti-inflammatory activity” as used in this application is defined as any measurable decrease in human tissue inflammation.

“Improved vision” as used in this application is defined as a patient's ability to read at least one additional line on a visual acuity eye chart test than the patient was previously able to read. In the standard eye chart test, a patient reads letters from a standard eye chart standing twenty feet away from the chart. The chart has different letters or symbols that a patient needs to discern from other letters or symbols. The letters or symbols on each line are of consistent size. Each line of the chart differs in size from other lines in the chart arranged in order from largest at the top of the chart to smallest at the bottom line of the chart. Each line of the eye chart is assigned a notation in the form of a fraction that represents visual acuity. The numerator is the distance in feet the patient is from the eye chart. The denominator represents the distance an eye with “normal” vision can read the same line.

Diagnostic Method

As noted above, the present invention is a method of screening a patient with macular edema associated with visual loss for implantation of a sustained release drug-delivery implant containing a steroid. The method according to one or more embodiments is described as follows.

a. Injecting a Steroid as a Bolus

A first steroid having anti-inflammatory activity is injected as a bolus into a patient's eye, particularly the posterior segment of a patient's eye. In one embodiment, the first steroid is a glucocorticoid steroid. In another embodiment, the first steroid is any steroid with anti-inflammatory activity selected from the group consisting of 21-acetoxypregnenolone, alclometasone, algestone, amcinonide, beclomethasone, betamethasone, budesonide, chloroprednisone, clobetasol, clobetasone, cloprednol, clocortolone, corticosterone, cortisone, cortivazol, deffazacort, desonide, desoximetasone, dexamethasone, diflucortolone, diruprednate, enoxolone, fluazacort, flucloronide, flumethasone, flunisolide, fluocinolone acetonide, fluocinonide, fluocortinbutyl, fluocortolone, fluorometholone, fluperolone acetate, fluprednidene acetate, fluprednisolone, flurandrenolide, formocortal, halcinonide, halometasone, halopredone acetate, hydrocortamate, diflorasone, hydrocortisone, hydrocortisone acetate, hydrocortisone phosphate, hydrocortisone 21-sodium succinate, hydrocortisone tebutate, mazipredone, medrysone, meprednisone, methylprednisolone, mometasone furoate, prednisolone sodium 21-m-sulfobenzoate, prednisolone 21-stearoylglycolate, prednisolone tebutate, prednisolone 21-trimethylacetate, prednisone, prednival, paramethasone, prednylidene, prednicarbate, prednylidene 21-diethylaminoacetate, prednisolone, prednisolone 21-diethylaminoacetate, tixocortol, triamcinolone, prednisolone sodium phosphate, progesterone, triamcinolone acetonide, prednisolone sodium succinate, triamcinolone benetonide, triamcinolone hexacetonide, and mixtures thereof.

In another embodiment, the first steroid is preferably selected from the group comprising betamethasone, hydrocortisone, hydrocortisone acetate, hydrocortisone phosphate, hydrocortisone 21-sodium succinate, hydrocortisone tebutate, triamcinolone acetonide, trimacinolone benetonide, trimacinolone hexacetonide, fluocinolone acetonide dexamethasone, dexamethasone sodium phosphate, prednisolone, prednisolone sodium phosphate, prednisolone acetate, fluorometholone acetate, dexamethasone, fluoromethalone, medrysone and mixtures thereof. In one preferred embodiment, the first steroid is triamcinolone acetonide.

Preferably, the amount of second steroid that is administered to the patient's eye in a sustained release drug-delivery implant is a minimum of about 0.01 mg and a maximum of about 25 mg. In one embodiment, the amount of the first steroid is a minimum of about 0.01 mg, about 0.02 mg, about 0.05 mg. In an embodiment, the amount of the first steroid is a maximum of 25 mg, about 20 mg, about 15 mg, about 10 mg, about 5 mg, about 3 mg, about 1 mg or 0.7 mg.

b. Identifying Candidate Patients

After a period of time passes to allow the medicament to relieve the symptoms associated with macular edema, the patient is observed to determine if the patient experiences a reduction in swelling in the retina associated with improved vision. A patient that satisfies this criteria is identified as a candidate patient for receiving a sustained release drug-delivery implant into the eye containing a second steroid.

Candidate patients are identified as patients that experience an improvement in vision and a reduction in swelling in the macula of the eye—preferably in the fovia.

In one embodiment, the reduction in swelling in the macula is measured by ocular coherence tomography. Ocular coherence tomography typically measures swelling of the fovia. Equipment and information on how to measure swelling in the macula and in particular the fovia by ocular coherence tomography is available from Carl Zeiss Opthalamic Systems, Inc. Dublin, Calif. See Sales Brochure for Stratus OCT, Carl Zeiss Opthalamic Systems Inc., Dublin Calif. (2002). See also Lee, et al., Optical coherence tomography for ophthalmic imaging: new technique delivers micron-scale resolution, Engineering in Medicine and Biology Magazine, Vol. 14(1), pp. 67-76 (January/February 1995).

The measurement of improved vision, in one embodiment, is an improvement in visual acuity by a factor of two lines on an eye chart test. Typically, the improvement in visual acuity is by a factor that is a minimum of three lines or four lines.

Furthermore, the evaluation measures whether a patient experiences an increase in intra-ocular pressure (IOP) side effects for the patient that experiences an increase in intraocular pressure during or after the step of injecting. “IOP side effect” as used herein is an increase in post-operative intraocular pressure.

In one embodiment, the measurement of intraocular pressure and hence “IOP side effects” requires the use of an applination tonometer. With the use of an applination tonometer intraocular pressure is measured before the operation to establish a baseline intraocular pressure. After the operation, intraocular pressure is measured, and preferably monitored over an evaluation period. The increase in the post-operative intraocular pressure over the baseline intraocular pressure is related to determining the IOP side effect. In one embodiment, the IOP side effect is determined by an increase in post-operative intraocular pressure minus baseline intraocular pressure that is a minimum of about 2 mm Hg. In another aspect, the increase in IOP side effects is an increase in intraocular pressure of a minimum of about 5 mm Hg, about 10 mm Hg, about 15 mm Hg, about 20 mm Hg or about 25 mm Hg minus the baseline intraocular pressure measured before the injection of the first steroid. Patients that have IOP side effects risk debilitating glaucoma. Accordingly, in one embodiment, patient's that have IOP side effects are not recommended for treatment with a sustained release drug-delivery implant.

The evaluation period for testing improved vision, reduction in swelling and IOP side effects is a maximum period of 90 days after the injection. Typically, the evaluation period for testing improved vision, reduction in swelling and IOP side effects are measured for a period having a minimum of about one day, about two days, about five days and a maximum of about 90 days, about 60 days, about 45 days, about 30 days, about 21 days or about 14 days. “Day” as used herein refers to a 24-hour period.

c. Patient Treatment

Typically, patients identified as candidate patients are recommended to receive a sustained release drug-delivery implant containing a second steroid.

Sustained release drug delivery implants for treatment of macular edema are typically inserted into the posterior chamber of a patient's eye. They exist in several forms, including but not limited to solid drug encapsulated implants, liposomes, microspheres, microcapsules and nanoparticles. Sustained release drug-delivery implants, their process for manufacture and methods of use are described in the following patents, the disclosures of which are incorporated herein by reference: US 2002/0086051A1 (drug-delivery implant); US 2002/0106395A1 (drug-delivery implant); US 2002/0110591A1 (drug-delivery implant); US 2002/0110592A1 (drug-delivery implant); US 2002/0110635A1 (drug-delivery implant); U.S. Pat. No. 4,853,224 (microcapsule implant); U.S. Pat. No. 4,997,652 (liposomes); U.S. Pat. No. 5,378,475; U.S. Pat. No. 5,773,019; U.S. Pat. No. 5,869,079 (implanted biodegradable drug mixture); U.S. Pat. No. 5,902,598; U.S. Pat. No. 6,001,386; U.S. Pat. No. 6,217,895; U.S. Pat. No. 6,331,313; U.S. Pat. No. 6,369,116 (implanted biodegradable drug mixture); U.S. Pat. No. 6,375,972; U.S. patent application Ser. No. 10/403,421, filed Mar. 28, 2003; and U.S. patent application Ser. No. 10/610,063, filed Jun. 30, 2003.

In one embodiment, the sustained release drug delivery implant delivers a second steroid that reduces swelling of retinal tissue when inserted into the candidate patient's eye. In one embodiment, the second steroid is a glucocorticoid steroid. In one preferred embodiment, the steroid is selected from the group consisting of 21-acetoxypregnenolone, alclometasone, algestone, amcinonide, beclomethasone, betamethasone, budesonide, chloroprednisone, clobetasol, clobetasone, cloprednol, clocortolone, corticosterone, cortisone, cortivazol, deffazacort, desonide, desoximetasone, dexamethasone, diflucortolone, diruprednate, enoxolone, fluazacort, flucloronide, flumethasone, flunisolide, fluocinolone acetonide, fluocinonide, fluocortinbutyl, fluocortolone, fluorometholone, fluperolone acetate, fluprednidene acetate, fluprednisolone, flurandrenolide, formocortal, halcinonide, halometasone, halopredone acetate, hydrocortamate, diflorasone, hydrocortisone, hydrocortisone acetate, hydrocortisone phosphate, hydrocortisone 21-sodium succinate, hydrocortisone tebutate, mazipredone, medrysone, meprednisone, methylprednisolone, mometasone furoate, prednisolone sodium 21-m-sulfobenzoate, prednisolone 21-stearoylglycolate, prednisolone tebutate, prednisolone 2 1-trimethylacetate, prednisone, prednival, paramethasone, prednylidene, prednicarbate, prednylidene 21-diethylaminoacetate, prednisolone, prednisolone 21-diethylaminoacetate, tixocortol, triamcinolone, prednisolone sodium phosphate, triamcinolone acetonide, prednisolone sodium succinate, triamcinolone benetonide, triamcinolone hexacetonide, and mixtures thereof.

More preferably, the second steroid is selected from the group comprising betamethasone, hydrocortisone, hydrocortisone acetate, hydrocortisone phosphate, hydrocortisone 21-sodium succinate, hydrocortisone tebutate, triamcinolone acetonide, trimacinolone benetonide, trimacinolone hexacetonide, fluocinolone acetonide dexamethasone, dexamethasone sodium phosphate, prednisolone, prednisolone sodium phosphate, prednisolone acetate, fluorometholone acetate, dexamethasone, fluoromethalone, medrysone and mixtures thereof, according to one embodiment. In one embodiment, the second steroid is fluocinolone acetonide.

Preferably, the amount of second steroid that is administered to the patient's eye in a sustained release drug-delivery implant is a minimum of about 0.01 mg and a maximum of about 25 mg. In one embodiment, the amount of the first steroid is a minimum of about 0.01 mg, about 0.02 mg, about 0.05 mg. In an embodiment, the amount of the first steroid is a maximum of 25 mg, about 20 mg, about 15 mg, about 10 mg, about 5 mg, about 3 mg, about 1 mg or 0.7 mg.

Product, System or Kit

One embodiment of the present invention is a kit for screening a patient with macular edema for use of a sustained release drug-delivery implant containing a first steroid. The kit comprises a fluid injection device having a fluid reservoir containing a first steroid, for example triamcinolone acetonide, and a cannula that is configured to be inserted into the vitreous cavity of a patient's eye. The fluid injection device also has a compression device that is configured to move the first steroid from the reservoir through the cannula. Additionally, the kit comprises instructions to inject a bolus of the first steroid into a patient's eye and identify a patient that has a reduction in swelling of tissue in the eye (typically the macula, preferably the fovia) and improved vision as a candidate patient for implantation of a sustained release, drug-delivery implant that contains a steroid with anti-inflammatory properties.

In one aspect of the invention, the instructions include direction to measure swelling in the macula by ocular coherence tomography. Generally, the instructions recommend the candidate patient to receive a drug-delivery implant containing a glucocorticoid steroid.

In another aspect of the invention, the instructions include directions on identifying IOP side effects for the patient that experiences an increase in intraocular pressure during or after the step of injecting. Typically, the IOP side effects are measured with an applination tonometer and the IOP side effects are measured according to any of the methods and embodiments described above. The IOP side effects are measured by an increase in intraocular pressure of a minimum of about 2 mm Hg or any other threshold described above.

The improvement in vision is measured by visual acuity test by a factor of at least one line, preferably two or three lines, most preferably four lines.

According to one feature of the present invention, the kit instructs the injection of a first steroid to determine if the patient is a candidate patient. Typically, the first steroid is a glucocorticoid steroid. In one embodiment, the first steroid is a steroid selected from the group consisting of 21-acetoxypregnenolone, alclometasone, algestone, amcinonide, beclomethasone, betamethasone, budesonide, chloroprednisone, clobetasol, clobetasone, cloprednol, clocortolone, corticosterone, cortisone, cortivazol, deffazacort, desonide, desoximetasone, dexamethasone, diflucortolone, diruprednate, enoxolone, fluazacort, flucloronide, flumethasone, flunisolide, fluocinolone acetonide, fluocinonide, fluocortinbutyl, fluocortolone, fluorometholone, fluperolone acetate, fluprednidene acetate, fluprednisolone, flurandrenolide, formocortal, halcinonide, halometasone, halopredone acetate, hydrocortamate, diflorasone, hydrocortisone, hydrocortisone acetate, hydrocortisone phosphate, hydrocortisone 21-sodium succinate, hydrocortisone tebutate, mazipredone, medrysone, meprednisone, methylprednisolone, mometasone furoate, prednisolone sodium 21-m-sulfobenzoate, prednisolone 21-stearoylglycolate, prednisolone tebutate, prednisolone 21-trimethylacetate, prednisone, prednival, paramethasone, prednylidene, prednicarbate, prednylidene 21-diethylaminoacetate, prednisolone, prednisolone 21-diethylaminoacetate, tixocortol, triamcinolone, prednisolone sodium phosphate, triamcinolone acetonide, prednisolone sodium succinate, triamcinolone benetonide, triamcinolone hexacetonide, and mixtures thereof.

The first steroid, in another embodiment is selected from the group comprising betamethasone, hydrocortisone, hydrocortisone acetate, hydrocortisone phosphate, hydrocortisone 21-sodium succinate, hydrocortisone tebutate, triamcinolone acetonide, trimacinolone benetonide, trimacinolone hexacetonide, fluocinolone acetonide dexamethasone, dexamethasone sodium phosphate, prednisolone, prednisolone sodium phosphate, prednisolone acetate, fluorometholone acetate, dexamethasone, fluoromethalone, medrysone and mixtures thereof. Preferably the first steroid is trimacinolone acetonide.

Although several specific embodiments have been depicted and described in detail, it will be apparent to those skilled in the relevant art that the specification is made without the intention of limiting the scope of the invention and that various modifications, additions, substitutions, and the like can be made without departing from the spirit of the invention are therefore considered to be within the scope of the invention as defined in the claims which follow.

1. A method for screening a patient with macular edema associated with visual loss for implantation of a sustained release drug-delivery implant, the method comprising the steps of:
(a) injecting a first steroid having anti-inflammatory activity as a bolus into a patient's eye; and
(b) identifying a patient that experiences a reduction in swelling in the retina associated with improved vision as a candidate patient that would benefit from the implantation of a sustained release drug-delivery implant into the eye containing a second steroid.

2. The method of claim 1, wherein the step of identifying identifies patients with a reduction in swelling in the macula of the eye.

3. The method of claim 2, wherein the step of identifying identifies patients with a reduction in swelling in the fovia.

4. The method of claim 2, wherein the swelling in the macula of the eye is measured by ocular coherence tomography.

5. The method of claim 1, further comprising the step of recommending a patient that is a candidate patient to receive a sustained release drug-delivery implant containing the second steroid.

6. The method of claim 1, wherein the first steroid is a glucocorticoid steroid.

7. The method of claim 6, wherein the first steroid is any steroid with anti-inflammatory activity selected from the group consisting of 21-acetoxypregnenolone, alclometasone, algestone, amcinonide, beclomethasone, betamethasone, budesonide, chloroprednisone, clobetasol, clobetasone, cloprednol, clocortolone, corticosterone, cortisone, cortivazol, deffazacort, desonide, desoximetasone, dexamethasone, diflucortolone, diruprednate, enoxolone, fluazacort, flucloronide, flumethasone, flunisolide, fluocinolone acetonide, fluocinonide, fluocortinbutyl, fluocortolone, fluorometholone, fluperolone acetate, fluprednidene acetate, fluprednisolone, flurandrenolide, formocortal, halcinonide, halometasone, halopredone acetate, hydrocortamate, diflorasone, hydrocortisone, hydrocortisone acetate, hydrocortisone phosphate, hydrocortisone 21-sodium succinate, hydrocortisone tebutate, mazipredone, medrysone, meprednisone, methylprednisolone, mometasone furoate, prednisolone sodium 21-m-sulfobenzoate, prednisolone 21-stearoylglycolate, prednisolone tebutate, prednisolone 21-trimethylacetate, prednisone, prednival, paramethasone, prednylidene, prednicarbate, prednylidene 21-diethylaminoacetate, prednisolone, prednisolone 21-diethylaminoacetate, tixocortol, triamcinolone, prednisolone sodium phosphate, progesterone, triamcinolone acetonide, prednisolone sodium succinate, triamcinolone benetonide, triamcinolone hexacetonide and mixtures thereof.

8. The method of claim 6, wherein the first steroid is selected from the group comprising betamethasone, hydrocortisone, hydrocortisone acetate, hydrocortisone phosphate, hydrocortisone 21-sodium succinate, hydrocortisone tebutate, triamcinolone acetonide, trimacinolone benetonide, trimacinolone hexacetonide, fluocinolone acetonide dexamethasone, dexamethasone sodium phosphate, prednisolone, prednisolone sodium phosphate, prednisolone acetate, fluorometholone acetate, dexamethasone, fluoromethalone, medrysone and mixtures thereof.

9. The method of claim 6, wherein the first steroid is triamcinolone acetonide.

10. The method of claim 1, wherein the second steroid is a glucocorticoid steroid.

11. The method of claim 10, wherein the second steroid is any steroid with anti-inflammatory activity selected from the group consisting of 21-acetoxypregnenolone, alclometasone, algestone, amcinonide, beclomethasone, betamethasone, budesonide, chloroprednisone, clobetasol, clobetasone, cloprednol, clocortolone, corticosterone, cortisone, cortivazol, deffazacort, desonide, desoximetasone, dexamethasone, diflucortolone, diruprednate, enoxolone, fluazacort, flucloronide, flumethasone, flunisolide, fluocinolone acetonide, fluocinonide, fluocortinbutyl, fluocortolone, fluorometholone, fluperolone acetate, fluprednidene acetate, fluprednisolone, flurandrenolide, formocortal, halcinonide, halometasone, halopredone acetate, hydrocortamate, diflorasone, hydrocortisone, hydrocortisone acetate, hydrocortisone phosphate, hydrocortisone 21-sodium succinate, hydrocortisone tebutate, mazipredone, medrysone, meprednisone, methylprednisolone, mometasone furoate, prednisolone sodium 21-m-sulfobenzoate, prednisolone 21-stearoylglycolate, prednisolone tebutate, prednisolone 21-trimethylacetate, prednisone, prednival, paramethasone, prednylidene, prednicarbate, prednylidene 21-diethylaminoacetate, prednisolone, prednisolone 21-diethylaminoacetate, tixocortol, triamcinolone, prednisolone sodium phosphate, triamcinolone acetonide, prednisolone sodium succinate, triamcinolone benetonide, triamcinolone hexacetonide and mixtures thereof.

12. The method of claim 10, wherein the second steroid is selected from the group comprising betamethasone, hydrocortisone, hydrocortisone acetate, hydrocortisone phosphate, hydrocortisone 21-sodium succinate, hydrocortisone tebutate, triamcinolone acetonide, trimacinolone benetonide, trimacinolone hexacetonide, fluocinolone acetonide dexamethasone, dexamethasone sodium phosphate, prednisolone, prednisolone sodium phosphate, prednisolone acetate, fluorometholone acetate, dexamethasone, fluoromethalone, medrysone and mixtures thereof.

13. The method of claim 10, wherein the second steroid is fluocinolone acetonide.

14. The method of claim 1, wherein the amount of the first steroid is a minimum of about 0.01 mg and a maximum of about 25 mg.

15. The method of claim 1, wherein the amount of the second steroid is a minimum of about 0.01 mg and a maximum of about 25 mg.

16. The method of claim 1, further comprising identifying IOP side effects for the patient that experiences an increase in intraocular pressure during or after the step of injecting.

17. The method of claim 16, wherein the IOP side effects are measure for a maximum period of 90 days after the injection.

18. The method of claim 16, wherein the IOP side effects are measured with an applination tonometer.

19. The method of claim 16, wherein the IOP side effect is an increase in intraocular pressure of a minimum of about 2 mm Hg.

20. The method of claim 1, wherein the sustained release drug-delivery implant is selected from the group comprising solid drug encapsulated implants, liposomes, microspheres, microcapsules and nanoparticles.

21. The method of claim 1, wherein the improved vision is an improvement in visual acuity by a factor that is a minimum of one line.

22. A method of treating a patient with macular edema comprising implanting a candidate patient identified by the method of claim 1, with a drug-delivery implant containing the second steroid.

23. The method of claim 22, wherein the step of identifying identifies patients with a reduction in swelling in the macula of the eye.

24. The method of claim 23, wherein the step of identifying identifies patients with a reduction in swelling in the fovia.

25. The method of claim 23, wherein the swelling in the macula of the eye is measured by ocular coherence tomography.

26. The method of claim 22, further comprising the step of recommending a patient that is a candidate patient to receive a sustained release drug-delivery implant containing the second steroid.

27. The method of claim 22, wherein the first steroid is a glucocorticoid steroid.

28. The method of claim 27, wherein the first steroid is any steroid with anti-inflammatory activity selected from the group consisting of 21-acetoxypregnenolone, alclometasone, algestone, amcinonide, beclomethasone, betamethasone, budesonide, chloroprednisone, clobetasol, clobetasone, cloprednol, clocortolone, corticosterone, cortisone, cortivazol, deffazacort, desonide, desoximetasone, dexamethasone, diflucortolone, diruprednate, enoxolone, fluazacort, flucloronide, flumethasone, flunisolide, fluocinolone acetonide, fluocinonide, fluocortinbutyl, fluocortolone, fluorometholone, fluperolone acetate, fluprednidene acetate, fluprednisolone, flurandrenolide, formocortal, halcinonide, halometasone, halopredone acetate, hydrocortamate, diflorasone, hydrocortisone, hydrocortisone acetate, hydrocortisone phosphate, hydrocortisone 21-sodium succinate, hydrocortisone tebutate, mazipredone, medrysone, meprednisone, methylprednisolone, mometasone furoate, prednisolone sodium 21-m-sulfobenzoate, prednisolone 21-stearoylglycolate, prednisolone tebutate, prednisolone 21-trimethylacetate, prednisone, prednival, paramethasone, prednylidene, prednicarbate, prednylidene 21-diethylaminoacetate, prednisolone, prednisolone 21-diethylaminoacetate, tixocortol, triamcinolone, prednisolone sodium phosphate, progesterone, triamcinolone acetonide, prednisolone sodium succinate, triamcinolone benetonide, triamcinolone hexacetonide and mixtures thereof.

29. The method of claim 27, wherein the first steroid is selected from the group comprising betamethasone, hydrocortisone, hydrocortisone acetate, hydrocortisone phosphate, hydrocortisone 21-sodium succinate, hydrocortisone tebutate, triamcinolone acetonide, triamcinolone benetonide, trimacinolone hexacetonide, fluocinolone acetonide dexamethasone, dexamethasone sodium phosphate, prednisolone, prednisolone sodium phosphate, prednisolone acetate, fluorometholone acetate, dexamethasone, fluoromethalone, medrysone and mixtures thereof.

30. The method of claim 27, wherein the first steroid is triamcinolone acetonide.

31. The method of claim 22, wherein the second steroid is a glucocorticoid steroid.

32. The method of claim 31, wherein the second steroid is any steroid with anti-inflammatory activity selected from the group consisting of 21-acetoxypregnenolone, alclometasone, algestone, amcinonide, beclomethasone, betamethasone, budesonide, chloroprednisone, clobetasol, clobetasone, cloprednol, clocortolone, corticosterone, cortisone, cortivazol, deffazacort, desonide, desoximetasone, dexamethasone, diflucortolone, diruprednate, enoxolone, fluazacort, flucloronide, flumethasone, flunisolide, fluocinolone acetonide, fluocinonide, fluocortinbutyl, fluocortolone, fluorometholone, fluperolone acetate, fluprednidene acetate, fluprednisolone, flurandrenolide, formocortal, halcinonide, halometasone, halopredone acetate, hydrocortamate, diflorasone, hydrocortisone, hydrocortisone acetate, hydrocortisone phosphate, hydrocortisone 21-sodium succinate, hydrocortisone tebutate, mazipredone, medrysone, meprednisone, methylprednisolone, mometasone furoate, prednisolone sodium 21-m-sulfobenzoate, prednisolone 21-stearoylglycolate, prednisolone tebutate, prednisolone 21-trimethylacetate, prednisone, prednival, paramethasone, prednylidene, prednicarbate, prednylidene 21-diethylaminoacetate, prednisolone, prednisolone 21-diethylaminoacetate, tixocortol, triamcinolone, prednisolone sodium phosphate, triamcinolone acetonide, prednisolone sodium succinate, triamcinolone benetonide, triamcinolone hexacetonide and mixtures thereof.

33. The method of claim 31, wherein the second steroid is selected from the group comprising betamethasone, hydrocortisone, hydrocortisone acetate, hydrocortisone phosphate, hydrocortisone 21-sodium succinate, hydrocortisone tebutate, triamcinolone acetonide, trimacinolone benetonide, trimacinolone hexacetonide, fluocinolone acetonide dexamethasone, dexamethasone sodium phosphate, prednisolone, prednisolone sodium phosphate, prednisolone acetate, fluorometholone acetate, dexamethasone, fluoromethalone, medrysone and mixtures thereof.

34. The method of claim 31, wherein the second steroid is fluocinolone acetonide.

35. The method of claim 22, wherein the amount of the first steroid is a minimum of about 0.01 mg and a maximum of about 25 mg.

36. The method of claim 22, wherein the amount of the second steroid is a minimum of about 0.01 mg and a maximum of about 25 mg.

37. The method of claim 22, further comprising identifying IOP side effects for the patient that experiences an increase in intraocular pressure during or after the step of injecting.

38. The method of claim 37, wherein the IOP side effects are measure for a maximum period of 90 days after the injection.

39. The method of claim 37, wherein the IOP side effects are measured with an applination tonometer.

40. The method of claim 37, wherein the IOP side effect is an increase in intraocular pressure of a minimum of about 2 mm Hg.

41. The method of claim 22, wherein the sustained release drug-delivery implant is selected from the group comprising solid drug encapsulated implants, liposomes, microspheres, microcapsules and nanoparticles.

42. The method of claim 22, wherein the improved vision is an improvement in visual acuity by a factor that is a minimum of one line.

43. A kit for screening a patient with macular edema for use of a sustained-release drug-delivery implant containing a first steroid, the kit comprising:
(a) a fluid injection device having a fluid reservoir containing a first steroid and a cannula that is configured to be inserted into the vitreous cavity of a patient's eye and a compression device that is configured to move the first steroid from the reservoir through the cannula;
(b) instructions to inject a bolus of the first steroid to a patient's eye and identify a patient that has a reduction in swelling of tissue in the eye and improved vision as a candidate patient for implantation of a sustained release drug-delivery implant that contains a steroid with anti-inflammatory properties.

44. The kit of claim 43, wherein the instructions instruct to identify patients with a reduction in swelling in the macula of the eye.

45. The kit of claim 44, wherein the instructions instruct to identify patients with a reduction in swelling in the fovia.

46. The kit of claim 44, wherein the instructions instruct to measure swelling in the macula by ocular coherence tomography.

47. The kit of claim 43, wherein the instructions recommend the candidate patient to receive a drug-delivery implant containing a glucocorticoid steroid.

48. The kit of claim 47, wherein the instructions recommend the candidate patient to receive a drug-delivery implant containing a steroid selected from the group consisting of 21-acetoxypregnenolone, alclometasone, algestone, amcinonide, beclomethasone, betamethasone, budesonide, chloroprednisone, clobetasol, clobetasone, cloprednol, clocortolone, corticosterone, cortisone, cortivazol, deffazacort, desonide, desoximetasone, dexamethasone, diflucortolone, diruprednate, enoxolone, fluazacort, flucloronide, flumethasone, flunisolide, fluocinolone acetonide, fluocinonide, fluocortinbutyl, fluocortolone, fluorometholone, fluperolone acetate, fluprednidene acetate, fluprednisolone, flurandrenolide, formocortal, halcinonide, halometasone, halopredone acetate, hydrocortamate, diflorasone, hydrocortisone, hydrocortisone acetate, hydrocortisone phosphate, hydrocortisone 21-sodium succinate, hydrocortisone tebutate, mazipredone, medrysone, meprednisone, methylprednisolone, mometasone furoate, prednisolone sodium 21-m-sulfobenzoate, prednisolone 21-stearoylglycolate, prednisolone tebutate, prednisolone 21-trimethylacetate, prednisone, prednival, paramethasone, prednylidene, prednicarbate, prednylidene 21-diethylaminoacetate, prednisolone, prednisolone 21-diethylaminoacetate, tixocortol, triamcinolone, prednisolone sodium phosphate, progesterone, triamcinolone acetonide, prednisolone sodium succinate, triamcinolone benetonide, triamcinolone hexacetonide and mixtures thereof.

49. The kit of claim 47, wherein the instructions recommend the candidate patient to receive a drug-delivery implant containing a steroid selected from the group comprising betamethasone, hydrocortisone, hydrocortisone acetate, hydrocortisone phosphate, hydrocortisone 21-sodium succinate, hydrocortisone tebutate, triamcinolone acetonide, trimacinolone benetonide, trimacinolone hexacetonide, fluocinolone acetonide dexamethasone, dexamethasone sodium phosphate, prednisolone, prednisolone sodium phosphate, prednisolone acetate, fluorometholone acetate, dexamethasone, fluoromethalone, medrysone and mixtures thereof.

50. The kit of claim 47, wherein the instructions recommend a patient to receive a drug-delivery implant containing fluocinolone acetonide.

51. The kit of claim 44, wherein the first steroid is a glucocorticoid steroid.

52. The kit of claim 51, wherein the first steroid is a steroid selected from the group consisting of 21-acetoxypregnenolone, alclometasone, algestone, amcinonide, beclomethasone, betamethasone, budesonide, chloroprednisone, clobetasol, clobetasone, cloprednol, clocortolone, corticosterone, cortisone, cortivazol, deffazacort, desonide, desoximetasone, dexamethasone, diflucortolone, diruprednate, enoxolone, fluazacort, flucloronide, flumethasone, flunisolide, fluocinolone acetonide, fluocinonide, fluocortinbutyl, fluocortolone, fluorometholone, fluperolone acetate, fluprednidene acetate, fluprednisolone, flurandrenolide, formocortal, halcinonide, halometasone, halopredone acetate, hydrocortamate, diflorasone, hydrocortisone, hydrocortisone acetate, hydrocortisone phosphate, hydrocortisone 21-sodium succinate, hydrocortisone tebutate, mazipredone, medrysone, meprednisone, methylprednisolone, mometasone furoate, prednisolone sodium 21-m-sulfobenzoate, prednisolone 21-stearoylglycolate, prednisolone tebutate, prednisolone 21-trimethylacetate, prednisone, prednival, paramethasone, prednylidene, prednicarbate, prednylidene 21-diethylaminoacetate, prednisolone, prednisolone 21-diethylaminoacetate, tixocortol, triamcinolone, prednisolone sodium phosphate, triamcinolone acetonide, prednisolone sodium succinate, triamcinolone benetonide, triamcinolone hexacetonide and mixtures thereof.

53. The kit of claim 51, wherein the first steroid is selected from the group comprising betamethasone, hydrocortisone, hydrocortisone acetate, hydrocortisone phosphate, hydrocortisone 21-sodium succinate, hydrocortisone tebutate, triamcinolone acetonide, trimacinolone benetonide, trimacinolone hexacetonide, fluocinolone acetonide dexamethasone, dexamethasone sodium phosphate, prednisolone, prednisolone sodium phosphate, prednisolone acetate, fluorometholone acetate, dexamethasone, fluoromethalone, medrysone and mixtures.

54. The kit of claim 51, wherein the first steroid is trimacinolone acetonide.

55. The kit of claim 44, wherein the instructions recommend injection of a minimum of about 0.01 mg and a maximum of about 25 mg of the first steroid.

56. The kit of claim 44, wherein the instruction instruct identifying IOP side effects for the patient that experiences an increase in intraocular pressure during or after the step of injecting.

57. The kit of claim 56, wherein the IOP side effects are measured for a maximum period of 90 days after the injection.

58. The kit of claim 56, wherein the IOP side effects are measured with an applination tonometer.

59. The kit of claim 56, wherein the IOP side effect is an increase in intraocular pressure of a minimum of about 2 mm Hg.

60. The kit of claim 44, wherein the improved vision is an improvement in visual acuity by a factor that is a minimum of one line.
